(19) United States
(12) Patent Application Publication
(10) Pub. No.: US 2019/0350851 A1
(43) Pub. Date: Nov. 21, 2019

(54) DOSE RESPONSE, SURFACE MODIFIED NANOTUBES

(71) Applicant: MOLECULAR REBAR DESIGN, LLC, Austin, TX (US)

(72) Inventors: Aaron Wallace Tasset, Austin, TX (US); Milos Marinkovic, San Antonio, TX (US); Kurt W. Swogger, Austin, TX (US)

(73) Assignee: MOLECULAR REBAR DESIGN, LLC, Austin, TX (US)

(21) Appl. No.: 16/414,055

(22) Filed: May 16, 2019

Related U.S. Application Data
(60) Provisional application No. 62/672,453, filed on May 16, 2018.

(51) Int. Cl. A61K 9/00; A61K 47/10; A61K 47/02; A61K 47/18

(52) U.S. Cl. CPC A61K 9/0092; A61K 47/10; A61K 45/06; A61K 47/186; A61K 47/02

(57) ABSTRACT

Discrete, individualized carbon nanotubes having targeted, or selective, oxidation levels or content and a functionalized surface coating are claimed. Such carbon nanotubes can have little to no inner tube surface oxidation, or differing amounts and/or types of oxidation between the tubes' inner and outer surfaces. These new discrete carbon nanotubes are useful for delivery and controlled release of drugs, chemicals, compounds, small molecules, oligonucleotides, peptides, proteins, enzymes, macromolecular gene-editing assemblies, other biologics and combinations of thereof. The functionalized surface coating may be utilized to preferentially direct the nanotubes to particular tissues, organs or regions of the body for controlled delivery and or release of a payload molecule.

### CROSS REFERENCE TO RELATED APPLICATIONS

The present application is related to U.S. application Ser. No. 62/319,599 filed on Apr. 7, 2016 and Ser. No. 62/424,606 filed Nov. 21, 2016, the entire disclosures of which are incorporated herein by reference. This application is also related to U.S. application Ser. No. 14/628,248 filed Feb. 21, 2015, as well as Ser. No. 13/164,456, filed Jun. 20, 2011, and their progeny; and Ser. No. 13/140,029, filed Aug. 9, 2011 and its progeny, the entire disclosures of which are incorporated herein by reference. This application claims benefit U.S. Provisional Application No. 62/672,453, filed May 16, 2018, the entire disclosure of which is incorporated herein by reference.

### FIELD OF INVENTION

The present application is directed to novel carbon nanotube compositions with functional coatings that permit medical or industrial applications using unique, discrete carbon nanotubes. The present application also encompasses the preparation of such carbon nanotubes.

### BACKGROUND AND SUMMARY OF THE INVENTION

Carbon nanotubes can be classified by the number of walls in the tube; single-wall, double wall and multiwall. Carbon nanotubes are typically manufactured as agglomerated nanotube balls, bundles or forests attached to substrates. The use of carbon nanotubes as a delivery device for drugs and small molecules is an area in which carbon nanotubes are predicted to have significant utility. However, utilization of carbon nanotubes in these applications has been hampered due to the general inability to reliably produce individualized carbon nanotubes and the ability to disperse the individualized carbon nanotubes biological environments such as blood or tissue. Bosnyak et al., in various patent applications (e.g., US 2012-0183770 A1 and US 2011-0294013 A1), have made discrete carbon nanotubes through judicious and substantially simultaneous use of oxidation and shear forces, thereby oxidizing both the inner and outer surface of the nanotubes, typically to approximately the same oxidation level on the inner and outer surfaces, resulting in individual or discrete tubes.

The present inventions can utilize the tubes of those earlier Bosnyak et al. applications and disclosures, but also uses new targeted oxidation discrete tubes. The present invention describes a composition of discrete, individualized carbon nanotubes having targeted, or selective, oxidation levels and/or oxygen content on the exterior and/or interior of the tube walls. Such novel carbon nanotubes can have little to no inner tube surface oxidation, or differing amounts and/or types of oxidation between the tubes' inner and outer surfaces. In certain embodiments, drugs, chemicals, compounds, and/or small molecules, biologics and/or complexes thereof may be loaded onto the exterior and/or into the interior of the nanotubes for delivery and/or controlled release.

One embodiment of the present invention is a composition comprising a plurality of discrete carbon nanotubes, wherein the discrete carbon nanotubes comprise an interior and exterior surface, each surface comprising an interior surface oxidized species content and an exterior surface oxidized species content, wherein the interior surface oxidized species content differs from the exterior surface oxidized species content by at least 20%, and as high as about 80% about 90%, about 95%, about 99%, about 99.5% or about 100%, preferably wherein the interior surface oxidized species content is less than the exterior surface oxidized species content.

The interior surface oxidized species content can be up to 3 weight percent relative to carbon nanotube weight, preferably from about 0.01 to about 3 weight percent relative to carbon nanotube weight, more preferably from about 0.01 to about 2, most preferably from about 0.01 to about 1. Especially preferred interior surface oxidized species content is from zero to about 0.01 weight percent relative to carbon nanotube weight.

The exterior surface oxidized species content can be from about 1 to about 6 weight percent relative to carbon nanotube weight, preferably from about 1 to about 4, more preferably from about 2 to about 3 weight percent relative to carbon nanotube weight. This is determined by comparing the exterior oxidized species content for a given plurality of nanotubes against the total weight of that plurality of nanotubes.

The interior and exterior surface oxidized species content totals can be from about 1 to about 9 weight percent relative to carbon nanotube weight.

The discrete carbon nanotubes of any composition embodiment herein may comprise a plurality of open ended tubes. The discrete carbon nanotubes of any composition embodiment herein are preferred wherein the inner and outer surface oxidation difference is at least about 0.2 weight percent.

The compositions described herein can be used as an ion or chemical transport. Various species or classes of compounds, drugs, chemicals, and/or small molecules, biologics and/or complexes thereof which demonstrate this ion or chemical transport effect can be used, including ionic, some non-ionic compounds, hydrophobic and/or hydrophilic compounds.

The compositions described herein can be used as storage for various organic or inorganic materials and their subsequent slow release.

The compositions comprising the novel discrete oxidized carbon nanotubes may also be used as a component in, or as, an imaging, sensor or diagnostic tool.

The compositions disclosed herein can also be used as a component in, or as, drug delivery or controlled release formulations.

The compositions disclosed herein can also be used as a component in, or as, a molecular delivery system to cells, including delivery all types of small molecules, surfactants, polymers, composites, organic and inorganic nanoparticles, proteins, peptides, nucleic acids, oligonucleotides, carbohydrates, lipids, glycosaminoglycans, proteoglycans, glycoproteins, steroids, antibodies, growth factors, viral vectors, genetic materials and gene-editing complexes, and combinations thereof.

The compositions disclosed herein can also be used as a component in, or as, a biomolecular delivery system to the extracellular environment, trans-membrane transport, transport into the internal cellular environment (cytoplasm) and/or transport to the cellular nucleus.

The compositions disclosed herein can also be used as a component in, or as, a biomolecular delivery system for all types of single-cell and multicellular organisms consisting of eukaryotic and/or prokaryotic cells, including mammalian, fungal, plant and bacterial cells, bacterial or plant organisms.

The compositions disclosed herein can also be used as a component in, or as, a biomolecular delivery system whereas the predominance of a delivery mechanism from [0014] may be controlled by selection of surface coatings and physical and chemical properties of the materials.

The compositions disclosed herein can also be used as a component in, or as, a biomolecular delivery system targeting specific organs or tissues within an organism and/or cell-types within organs and tissues, by selection of surface coatings and physical and chemical properties of the carbon nanotubes.

The present inventions may also comprise carbon nanotubes with surface coatings. A surface coating may be functionalized, may be covalently linked to the nanotube surface and/or may be non-covalently bound through hydrophobic, hydrophilic, amphiphilic and/or electrostatic interactions. The present application discloses a novel manufacturing process for surface coated carbon nanotubes.

The present application also discloses a novel use for the carbon nanotubes in nanotube-mediated controlled delivery of drugs, chemicals, compounds, and/or small molecules, oligonucleotides, peptide, proteins, enzymes, antibodies, other types of biologics and/or complexes thereof. Typically, small molecule drugs, like Doxorubicin (“DOX”), or biologics such as siRNA, are cleared from the body or rapidly metabolized. In some embodiments, by binding a drug or small molecule and/or biologic to the surface of a nanotube or to a surface coating, such as, for example, poly ethylene glycol (“PEG”), this rapid clearance may be slowed by allowing more of the drug to reach the desired target and remaining at the target site. Typical targets include tissue and organ-specific targets such as liver, bone, and blood and/or resident cell-types such as T lymphocytes, dendritic cells, macrophages, Kupffer cells and osteoblasts.

The biodistribution of carbon nanotubes to tissues and residing cells can be controlled via selection and functionalization of the surface coating. In certain embodiments, the drug-loaded nanotubes may be preferentially directed to either bone or liver which allows for increased control of the amount of drug getting to a target site. The disclosed methods and techniques may also be used to address tissue and organic-specific delivery of drugs, chemicals, compounds, nanoparticles, and/or small molecules, biologics and/or complexes thereof for use in treatment, therapy, scanning, imaging, and/or diagnostics and/or manufacturing.

Another aspect of the disclosed inventions is the controlled release of drugs, chemicals, compounds, nanoparticles, and/or small molecules, biologics and/or complexes thereof which may be bound to a nanotube surface coating. Nanotube surface coatings may include molecules which undergo chemical and/or physical changes in response to changes in environmental conditions, including but not limited to temperature, ionic concentration, and/or pH. Such molecules may be incorporated, either directly through covalent attachment or indirectly through ionic, hydrophilic, hydrophobic and electrostatic interactions onto the surface of nanotubes in order to further regulate delivery. As one of many examples, a pH-sensitive polymer which decomposes at acidic pH below 7.4 would allow for selective delivery of drugs to acidic environments such as tumor-like environments.

Nanotubes may include molecular surface coatings or payloads whose uptake and/or release from the nanotube may be controlled by competitive association of ionic or chemical or biomolecular species with the external and/or internal surfaces of the nanotube or the molecular surface coatings of payloads themselves. As one of many examples, differences in calcium concentration gradients may be used as means of controlling release of biomolecular payloads in systems where calcium ions above a threshold concentration compete with biomolecular surface coatings or payloads for occupying the nanotube surface, causing release of the biomolecular surface coating or payload.

Nanotubes may feature targeting agents and/or adjuvants, covalently or non-covalently associated with the external surfaces, internal surfaces and/or payload molecules associated with either, which alter biodistribution of nanotubes within organisms such that specific organs, tissues and/or cells residing therein are targeting for accumulation and/or clearance of the nanotubes. These targeting agents may be both inorganic and/or organic in nature, of components such as nanoparticles, small molecules, oligonucleotides, peptide, proteins, enzymes, antibodies, other types of biologics and/or complexes thereof.

Discrete nanotubes, covalently or non-covalently associated with molecular surface coatings and/or payloads, which enable accumulation and/or clearance from specific organs, tissues and/or cells, with approximately normal distributions of average lengths ranging from 800 nm to 10 nm may be preferred for greater (by mass) and more rapid uptake and/or clearance from various from organs, tissues and/or cells. Molecular surface coatings and/or payloads can consist of: ionic and non-ionic species, inorganic and organic nanoparticles, small molecules, polysaccharides, oligonucleotides, peptide, proteins, enzymes, antibodies, other types of biologics and/or complexes thereof. For example, a collection of intravenously administered, discrete nanotubes with an approximately normal size distribution averaging 450 nm is demonstrated to provide approximately 15% to 20% greater distribution to bone tissue (by mass fraction of total) and up to 39% lower distribution to liver (by mass fraction of total) relative to a population of discrete nanotubes with an approximately normal size distribution averaging 850 nm. In another example, a preferential distribution of nanotube length may be used in an application where distinct biological payloads on each of a nanotube are used to associate, bind to or other affect two distinct biological targets in tissues and/or cells, such as a therapeutic, imaging or sensing system in which a nanotube complexed with two different antibodies binds distinct surface proteins on cells.

In another aspect the invention is a composition comprising a plurality of functionalized discrete single-wall, double-wall, or multi-wall carbon nanotubes having an innermost wall and an outermost wall, the inner-most wall defining an interior cavity, and at least one type of payload molecule; wherein the functionalized discrete carbon nanotubes are open on at least one end; and wherein greater than about 30 weight percent of the at least one type of payload molecule is within the interior cavity of the discrete single-wall, double-wall or multi-wall carbon nanotubes.

The functionalizing groups and/or the surface coating are not limited to, but can be selected from, the group consisting of bio-compatible surfactants, ionic and zwitterionic moieties. Preferred bio-compatible surfactants include, but are not limited to, PLA (polylactic acid), PVOH (polyvinyl alcohol), PEO (polyethylene oxide), PGLA (polyglycolic acid), CMC (carboxymethyl cellulose), PVP polyvinylpyrrolidone, PAA polyacrylic acid, aminoacids, peptides, polysaccharides, carboxy betaine-based systems, phosphoryl choline-based systems, nucleic acids (e.g., DNA) and proteins (e.g., albumin). The open ended multi-wall discrete carbon nanotubes preferably comprise at least one end having attached thereto a bio-compatible polymer, amino acid, protein or peptide, enzyme, nucleic acid oligonucleotide, or complex thereof.

The attachment may be via covalent bonding, ionic bonding, hydrogen bonding or pi-pi bonding in nature. That is, “attached” as used herein may, depending on the context, include covalent bonding, ionic bonding, hydrogen bonding, pi-pi bonding, or other adherence, as well as, combinations thereof. The functionalized discrete carbon nanotubes can include at least one tissue-targeting moiety. Use of tissue-targeting moieties is known in the art to provide directed delivery of a drug to a particular tissue in vivo, such as a tumor tissue. The compositions may also be directed to certain cellular receptors, such as through receptor ligands attached to the functionalized carbon nanotube. In some disease states, such as but not limited to cancer, certain cellular receptors are either overexpressed or in a high-activity binding state. Direction of the compositions herein to cellular receptors advantageously provides a means of targeting a particular tissue or cell type. The at least one tissue-targeting moiety is selected from a group including, but not limited to, aptamers, nucleic acids, antibodies, antibody fragments, polysaccharides, peptides, proteins, hormones, receptor ligands, synthetic derivatives thereof, and combinations thereof. Various cellular recognition sites exist for these moieties, allowing for directed tissue targeting of the compositions.

At least one type of payload molecule is preferably at least partially released from the open ended multi-wall discrete carbon nanotubes by a mechanism comprising diffusion, electromagnetic radiation exposure (e.g., MRI (Magnetic Resonance Imaging)), local pH changes, electrolyte balance, or biological (e.g., enzymatic) digestion of the biopolymer coat.

The plurality of functionalized discrete open ended multi-wall carbon nanotubes preferably comprises nanotubes of varying lengths. The tube length distribution may be monomodal, bimodal or multimodal. For tube length distributions comprising at least 2 groups of lengths, preferred is wherein each group's tube length varies on average by at least about 10% from the other group's average tube length to control drug release rates. Different length distributions may contain different payload molecules, different targeting moieties, and/or different surface coatings.

The plurality of functionalized discrete open ended multi-wall carbon nanotubes comprises an average aspect ratio (Length/Diameter) of from about 25 to about 500, preferably 25-250 and most preferably 40-120.

The plurality of functionalized discrete open ended multi-wall carbon nanotubes can comprise 0.01 to 99.9% by weight of the composition, preferably 0.1 to 99%, more preferably 0.25 to 95% by weight of the composition.

Based on the desired rate of payload delivery 10% by weight or less of the discrete carbon nanotubes of the composition can comprise L/D of about 100 to 200 and about 30% or more of the discrete carbon nanotubes (known and referred to herein as Molecular Rebar (“MW”) of the composition can comprise L/D of 10 to 80, preferably 40 to 80. The L/D of the discrete carbon nanotubes can be a unimodal distribution, or a multimodal distribution (such as a bimodal distribution). The multimodal distributions can have evenly distributed ranges of aspect ratios (such as 50% of one L/D range and about 50% of another L/D range). The distributions can also be asymmetrical—meaning that a relatively small percent of discrete nanotubes can have a specific L/D while a greater amount can comprise another aspect ratio distribution.

The payload molecule is not limited to, but can be selected from, the group consisting of a drug molecule, a radiotracer molecule, a radiotherapy molecule, a nanoparticle, a diagnostic imaging molecule, a fluorescent tracer molecule, a peptide, a protein, an enzyme, a nucleic acid, an oligonucleotide, a genetic compound, an antibody, cellular components, other biologics and combinations thereof.

Exemplary types of payload molecules that may be covalently or non-covalently associated with the discrete functionalized carbon nanotubes disclosed herein may include, but are not limited to, proton pump inhibitors, H2-receptor antagonists, cytoprotectants, prostaglandin analogues, beta blockers, calcium channel blockers, diuretics, cardiac glycosides, antiarrhythmics, antianginals, vasoconstrictors, vasodilators, ACE inhibitors, angiotensin receptor blockers, alpha blockers, anticoagulants, antiplatelet drugs, fibrinolytics, hypolipidemic agents, statins, hypnotics, antipsychotics, antidepressants, monoamine oxidase inhibitors, selective serotonin reuptake inhibitors, antiemetics, anticonvulsants, anxiolytic, barbiturates, stimulants, amphetamines, benzodiazepines, dopamine antagonists, antihistamines, cholinergics, anticholinergics, emetics, cannabinoids, 5-HT antagonists, NSAIDs, opioids, bronchodilator, antiallergics, mucolytics, corticosteroids, beta-receptor antagonists, anticholinergics, steroids, androgens, antiandrogens, growth hormones, thyroid hormones, anti-thyroid drugs, vasopressin analogues, antibiotics, antifungals, antituberculous drugs, antimalarials, antiviral drugs, antiprotozoal drugs, radioprotectants, chemotherapy drugs, cytostatic drugs, cytotoxic drugs such as paclitaxel, and biologics, including peptides, proteins, such as antibodies and antibody fragments, as well as nucleic acids, including expression vectors, siRNAs, mRNAs, microRNAs, DNA plasmids, adjuvants, vaccines and the like.

In another aspect of the invention, a payload or drug molecule delivery system composition comprising discrete carbon nanotubes is disclosed, wherein at least a portion of discrete nanotubes have a ratio of number average value of ((tube contour length (TCL)):(tube end to end length (TEE))) of from about 1.1 to about 3, preferably from about 1.1 to about 2.8, more preferably from about 1.1 to about 2.4, most preferably from about 1.1 to about 2 and especially from about 1.2 to about 2.

Another aspect of the inventions is a payload or drug delivery system composition comprising a plurality of discrete carbon nanotubes wherein at least a portion of discrete nanotubes have a number average tube contour length (TCL) of at least 10% greater than, and up to about 300% of, a number average tube end to end length (TEE), wherein the number average TCL and TEE are obtained from the same batch of discrete carbon nanotubes.

In another aspect, in a payload or drug delivery system composition comprising a plurality of discrete carbon nanotubes having an average actual aspect ratio, of at least about 5% (volume) of the discrete carbon nanotubes having an apparent aspect ratio from about 50% to about 99% of the average actual aspect ratio of the discrete carbon nanotubes. The apparent aspect ratio can be from a low of about 60% or 70%, to as high as about 80%, 90%, about 99% of the actual aspect ratio.

The composition can have at least about 10% (volume), preferably 20%, more preferably 50%, most preferably 75%, and especially 95%, of the discrete carbon nanotubes that have an apparent aspect ratio from about 50% to about 99% of the actual aspect ratio of the discrete carbon nanotubes.

Another aspect of the invention is in a payload or drug delivery system composition comprising a plurality of discrete carbon nanotubes having a number average contour length TCL, the improvement comprising at least about 5% (volume) of the discrete carbon nanotubes have a number average end-to-end tube length TEE low value from about 50%, 60%, or 70% to a high value of about 80%, 90%, or 99% of the number average TCL.

The composition can have at least about 10% (volume), preferably 20%, more preferably 50%, most preferably 75%, and especially 95%, of the discrete carbon nanotubes have a number average TEE from about 50% to about 99% of the number average TCL of the discrete carbon nanotubes.

The at least a portion of discrete nanotubes can have a number average value (the ratio of discrete TCL to TEE) of about 1.1 to as high as about 3, is greater than 5% by number, preferably greater than 20% by number and most preferably greater than 50% by number of tubes.

Processes to make the discrete carbon nanotubes are also described herein. These additional (and optional) steps can be selected from adding the discrete carbon nanotubes to a material to react with the oxidized discrete carbon nanotubes, adding surfactants, and adding other molecules including drugs, chemicals, compounds, and/or small molecules.

For a more complete understanding of the present disclosure, and the advantages thereof, reference is now made to the following descriptions describing specific embodiments of the disclosure.

### BRIEF DESCRIPTION OF THE FIGURES

FIG. 1 depicts the change in the biodistribution of nanotubes in bone when the nanotubes are functionalized with different surfactants

FIG. 2 depicts the change in the biodistribution of nanotubes in liver when the nanotubes are functionalized with different surfactants

FIG. 3 depicts the change in the biodistribution of nanotubes in bone as a result of different lengths. Both nanotubes featured the same surfactant treatment.

FIG. 4 depicts the change in the biodistribution of nanotubes in liver as a result of different lengths. Both nanotubes featured the same surfactant treatment.

FIG. 5 describes the biodistribution data of a particular embodiment of the disclosed nanotubes based on the route of administration.

FIG. 6 describes the biological clearance data of a particular embodiment of the disclosed nanotubes.

### DETAILED DESCRIPTION

In the following description, certain details are set forth such as specific quantities, sizes, etc., so as to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless or essentially meaningless, the definition should be taken from Webster's Dictionary, 3rd Edition, 2009. Definitions and/or interpretations should not be incorporated from other patent applications, patents, or publications, related or not.

Functionalized carbon nanotubes of the present disclosure generally refer to the chemical modification of any of the carbon nanotube types described hereinabove. Such modifications can involve the nanotube ends, sidewalls inside and/or outside, or both. Chemical modifications may include, but are not limited to covalent bonding, ionic bonding, chemisorption, intercalation, surfactant interactions, polymer wrapping, cutting, solvation, and combinations thereof. In some embodiments, the carbon nanotubes may be functionalized before, during and after being individualized or exfoliated.

Any of the aspects disclosed in this application with discrete carbon nanotubes may also be modified within the spirit and scope of the disclosure to substitute other tubular and non-tubular nanostructures, including, for example, organic, inorganic, or mineral nanotubes, planar nanostructures, and/or other nanostructures. Inorganic or mineral nanotubes include, for example, silicon nanotubes, boron nitride nanotubes and carbon nanotubes having heteroatom substitution in the nanotube structure. The nanotubes may include or be associated with organic or inorganic elements such as, for example, carbon, silicon, boron and nitrogen. Association may be on the interior or exterior of the inorganic or mineral nanotubes via Van der Waals, ionic or covalent bonding to the nanotube surfaces. Planar nanostructures include substantially planar carbon compounds such as graphene and similar structures composed of or including silicon, boron nitride, and carbon structures having heteroatom substitution in the nanostructure. This planar nanostructure may include or be associated with organic or inorganic elements such as, for example, carbon, silicon, boron and nitrogen. Association may be on either or both surfaces of the planar nanostructure via Van der Waals, ionic or covalent bonding to the planar nanostructure surfaces. Other nanostructures include three dimensional carbon structures such as fullerenes and similar structures composed of or including silicon, boron nitride, and carbon structures having heteroatom substitution in the nanostructure. These other nanostructures may also include or be associated with organic or inorganic elements such as, for example, carbon, silicon, boron and nitrogen. Association may be on the interior or exterior of the nanostructure via Van der Waals, ionic or covalent bonding to the nanotube surfaces

In various embodiments, a plurality of carbon nanotubes is disclosed comprising single wall, double wall or multi wall carbon nanotubes having an aspect ratio of from about 10 to about 500, preferably from about 40 to about 200, and an overall (total) oxidation level of from about 1 weight percent to about 15 weight percent, preferably from about 1 weight percent to about 10 weight percent, more preferably from about 1 weight percent to 5 weight percent, more preferably from about 1 weight percent to 3 weight percent. The oxidation level is defined as the amount by weight of oxygenated species covalently bound to the carbon nanotube. The thermogravimetric method for the determination of the percent weight of oxygenated species on the carbon nanotube involves taking about 7-15 mg of the dried oxidized carbon nanotube and heating at 5° C./minute from 100 degrees centigrade to 700 degrees centigrade in a dry nitrogen atmosphere. The percentage weight loss from 200 to 600 degrees centigrade is taken as the percent weight loss of oxygenated species. The oxygenated species can also be quantified using Fourier transform infra-red spectroscopy, FTIR, particularly in the wavelength range 1730-1680 cm-1

The carbon nanotubes can have oxidation species comprising carboxylic acid or derivative carbonyl containing species and are essentially discrete individual nanotubes, not entangled as a mass. Typically, the amount of discrete carbon nanotubes after completing the process of oxidation and shear is by a far a majority (that is, a plurality) and can be as high as 70, 80, 90 or even 99 percent of discrete carbon nanotubes, with the remainder of the tubes still partially entangled in some form. Complete conversion (i.e., 100 percent) of the nanotubes to discrete individualized tubes is most preferred. The derivative carbonyl species can include phenols, ketones, quaternary amines, amides, esters, acyl halogens, monovalent metal salts and the like, and can vary between the inner and outer surfaces of the tubes.

For example, one type of acid can be used to oxidize the tubes exterior surfaces, followed by water washing and the induced shear, thereby breaking and separating the tubes. If desired, the formed discrete tubes, having essentially no (or zero) interior tube wall oxidation can be further oxidized with a different oxidizing agent, or even the same oxidizing agent as that used for the tubes' exterior wall surfaces at a different concentration, resulting in differing amounts—and/or differing types—of interior and surface oxidation.

The discrete carbon nanotubes (known and referred to herein as Molecular Rebar (“MW”) have oxidized species on the surface, also known herein as functionalized groups. In this disclosure, the amount of oxidation can be from about 1 to about 15% by weight of the dried carbon nanotubes. Oxidized species include but not limited to carboxylates, hydroxyls, lactones, and combinations thereof. The oxidized species can react advantageously with species such as, but not limited to, an acylchloride, epoxy, isocyanate, hydroxyl, or amine group. The Molecular Rebar may further comprise a biocompatible dispersing agent or surfactant, adhesively, ionically or covalently bonded to the Molecular Rebar surface. The biocompatible dispersing or surfactant molecule can be chosen such that the size of the surfactant molecule in the liquid media prevents it from entering within the discrete carbon nanotube. The selection of the minimum size of the surfactant molecule that cannot enter into a tube opening is related to the diameter of the tube opening and the hydrodynamic radius of the molecule in the liquid media.

Hydrodynamic radius, R_{H}, of polymer molecules in liquid media has been well-studied in the scientific literature, for example M. S. Ahmed, M. S. El-Aassar and J. W. Vanderhoff, ACS Symp. Series 240:77 (1983). Techniques to measure the radius of gyration commonly include viscometry and photon correlation spectroscopy. In the studies by Ahmed et al., the values of R_{H} of polyvinyl alcohol adsorbed onto polystyrene particles of diameter 190 nm in water were found to follow an equation R_{H}=0.03 Mw^{0.538}.

The size of the surfactant molecule that can disperse the discrete carbon nanotube in aqueous media and is not expected to be able to enter within the cavity of the open ended carbon nanotube is preferably greater than about 30,000 Daltons, more preferably greater than about 60,000 Daltons and most preferably greater than about 100,000 Daltons. An example of a biocompatible polymer that is of size that does not fit within carbon nanotubes with an internal diameter opening of 5 nm is polyvinyl alcohol of molecular weight about 61,000 Daltons, available as Mowiol 10-98, supplied by Kuraray.

TABLE 1
The Hydrodynamic Radius of Various Molecules
in Water for Various Molecules

| | Molecular Weight, | Hydrodynamic |
|---|---|---|
| Molecule | Daltons | Radius, nm |
| Niacin | 123 | 0.33 |
| Nicotine | 162 | 0.38 |
| Tryptophan | 204 | 0.43 |
| Scopolamine | 303 | 0.52 |
| Fentanyl | 332 | 0.54 |
| Desmopressin | 1069 | 0.88 |
| Insulin | 6000 | 2.1 |
| Cytochrome C | 11700 | 2.64 |
| Myoglobin | 15300 | 3.34 |
| Bovine serum albumin | 67000 | 7.0 |

The hydrodynamic radius, RH of single amino acids, small di- and tripeptides as well as denatured proteins fit an equation RH=0.027M^{0.5} nm. (J. Danielson. PhD. Thesis Stockholm University 2007). For PVOH this has been found to be RH=0.03M^{0.538}nm. It is recognized that the value of the hydrodynamic radius is also dependent on the solvent quality, i.e., RH will decrease for insulin in acid conditions versus neutral conditions. Likewise a change in temperature can also cause a change in values of RH. This change in hydrodynamic radius may be conducive to fit molecules within the interior cavity of the discrete carbon nanotube; then to change the liquid media environment and force expansion of the molecules' hydrodynamic radius and cause expulsion of the drug molecule from the interior cavity, one needs to change the environment, such as for example, by changing liquid media environment temperature, pH or both.

Single-wall and double-wall carbon nanotubes typically have internal diameters of about 0.9 to about 1.2 nm. Multi-wall carbon nanotubes typically have internal diameters from about 1.8 to about 50 nm. Molecules are considered unlikely to enter into open ended carbon nanotubes if their hydrodynamic radius is about 10% larger than that of the carbon nanotube opening. This means, for example looking at Table 1 (above), that insulin, with a hydrodynamic radius of 2.1 nm would not be able to enter inside an opened single wall or double wall carbon nanotube Likewise, bovine serum albumin with a hydrodynamic radius of 7 nm would not enter into an open ended multiwall carbon nanotube of internal diameter 5.5 nm. This means that the selection of the innermost wall diameter of the discrete carbon nanotube plays a key role in selecting the maximum size of molecule that can enter into the carbon nanotube.

The biocompatible dispersing or surfactant molecule can also be chosen to help solubilize a drug in an aqueous media such that the drug and surfactant conjugate can enter into an open ended nanotube, followed by the nanotube and contents being encapsulated with a larger biomolecule that cannot enter into the tube. The size of the surfactant molecule being able to enter into the nanotube is preferably less than about 10,000 Daltons, more preferably less than about 5,000 Daltons and most preferably less than about 2,000 Daltons. An example of this type of surfactant being able to enter into a multiwall nanotube is polyoxyethene sorbitan monostearate of molecular weight about 1,309 Daltons and is commercially available as Tween-60 (Tween is a registered Trademark of Croda International PLC). As a result of the aforementioned, discrete carbon nanotubes may result in advantageous drug transport properties.

The internal tube diameter of the open ended carbon nanotube can be selected to allow a maximum size of the drug molecule to enter within the tube. This can be useful to select a certain size molecule from a mixture of molecules of different sizes. Open ended carbon nanotubes of different internal diameter tubes and/or different lengths can be used to control the rate of drug delivery, or combinations of drug types or sizes. Discrete open ended carbon nanotubes of differing functionality can also be used to control the rate of release of the drug to the treatment site.

The discrete oxidized carbon nanotubes alternatively termed exfoliated carbon nanotubes, of the present disclosure can take advantage of properties such as electrical, thermal, physical and drug transport, offered by individual carbon nanotubes that are not apparent when the carbon nanotubes are aggregated into bundles. An example of properties offered by individual carbon nanotubes rather than bundled or associated carbon nanotubes would be to deliver drug concentrations more accurately and for individual carbon nanotubes to be preferentially oriented alongside cell walls or to enter within cells.

Discrete oxidized carbon nanotubes, alternatively termed exfoliated carbon nanotubes, are obtained from as-made bundled carbon nanotubes by methods disclosed in U.S. Ser. Nos. 13/164,456 and 13/140,029, the disclosures of which are incorporated herein by reference, are particularly useful in producing the discrete carbon nanotubes used in this invention. The bundled carbon nanotubes can be made from any known means such as, for example, chemical vapor deposition, laser ablation, and high pressure carbon monoxide synthesis. The bundled carbon nanotubes can be present in a variety of forms including, for example, soot, powder, fibers, and bucky paper. Furthermore, the bundled carbon nanotubes may be of any length, diameter, or chirality. Carbon nanotubes may be metallic, semi-metallic, semi-conducting, or non-metallic based on their chirality and number of walls. The discrete oxidized carbon nanotubes may include, for example, single-wall, double-wall carbon nanotubes, or multi-wall carbon nanotubes and combinations thereof. One of ordinary skill in the art will recognize that some of the specific aspects of this invention illustrated utilizing a particular type of carbon nanotube may be practiced equivalently within the spirit and scope of the disclosure utilizing other types of carbon nanotubes. However, for control of the desired structures of a plurality of discrete carbon nanotubes requires a specific control of chemistry, thermal and mechanical energy which varies according to the starting structure of the carbon nanotubes.

In particular for forming carbon nanotubes of this invention is the incorporation of a portion of structures called Stone-Wales defects which are the rearrangement of the six-membered rings of graphene into heptagon-pentagon pairs that fit within the hexagonal lattice of fused benzene rings constituting a wall of the carbon nanotubes. These Stone-Wales defects are useful to create sites of higher bond-strain energy for more facile oxidation of the graphene or carbon nanotube wall. These defects and other types of fused ring structures may also facilitate bending or curling along the length of the carbon nanotubes.

Stone-Wales defects are thought to be more prevalent at the end caps that allow higher degrees of curvature of the walls of carbon nanotubes. During oxidation the ends of the carbon nanotubes can be opened and also result in higher degrees of oxidation at the opened ends than along the walls. The higher degree of oxidation and hence higher polarity or hydrogen bonding at the ends of the tubes are thought useful to help increase the average contour length to end to end ratio where the tubes are present in less polar media such as oil. The ratio of the contour length to end to end distance can be advantageously controlled by the degree of thermodynamic interaction between the tubes and the medium. Surfactants and electrolytes can be usefully employed also to modify the thermodynamic interactions between the tubes and the medium of choice. Alternate means to influence the ratio of contour length to end to end ratio include the use of inorganic or ionic salts and organic containing functional groups that can be attached to or contacted with the tube surfaces.

Bundled Carbon Nanotubes

As manufactured carbon nanotubes are obtainable in the form of bundles or entangled agglomerates and can be obtained from different sources, such as CNano Technology, Nanocyl, Arkema, and Kumho Petrochemical, to make discrete carbon nanotubes. An acid solution, preferably nitric acid solution at greater than about 60 weight % concentration, more preferably above 65% nitric acid concentration, can be used to prepare the carbon nanotubes for later shear to make the discrete tubes. Mixed acid systems (e. g. nitric and sulfuric acid) as disclosed in US 2012-0183770 A1 and US 2011-0294013 A1, the disclosures of which are incorporated herein by reference, can be used to produce discrete, oxidized carbon nanotubes from as—made bundled or entangled carbon nanotubes. The carbon nanotubes may be used consistent with the methods described in U.S. Pat. No. 7,992,640; U.S. Application No. 2015/0368541; and U.S. Application No. 2014/0014586, all of which are incorporated herein by reference.

As-made carbon nanotubes using metal catalysts such as iron, aluminum or cobalt can retain a significant amount of the catalyst associated or entrapped within the carbon nanotube, as much as five weight percent or more. These residual metals can be deleterious in such applications as drug delivery, treatment, imaging, and/or diagnostics because of such residual metals may not be biocompatible. Furthermore, these divalent or multivalent metal ions can associate with carboxylic acid groups on the carbon nanotube and interfere with the discretization of the carbon nanotubes in subsequent dispersion processes. In other embodiments, the oxidized carbon nanotubes comprise a residual metal concentration of less than about 25,000 parts per million, ppm, and preferably less than about 5,000 parts per million. The metals composition and concentration can be conveniently determined using energy dispersive X-ray spectroscopy or thermogravimetric methods.

General Process to Produce Discrete Carbon Nanotubes Having Targeted Oxidation

A mixture of 0.5% to 5% carbon nanotubes, preferably 3%, by weight is prepared with CNano grade Flotube 9000 carbon nanotubes and 65% nitric acid. While stirring, the acid and carbon nanotube mixture is heated to 70 to 90 degrees C. for 2 to 4 hours. The formed oxidized carbon nanotubes are then isolated from the acid mixture. Several methods can be used to isolate the oxidized carbon nanotubes, including but not limited to centrifugation, filtration, mechanical expression, decanting and other solid—liquid separation techniques. The residual acid is then removed by washing the oxidized carbon nanotubes with an aqueous medium such as water, preferably deionized water, to a pH of 3 to 4. The carbon nanotubes are then suspended in water at a concentration of 0.5% to 4%, preferably 1.5% by weight. The solution is subjected to intensely disruptive forces generated by shear (turbulent) and/or cavitation with process equipment capable of producing energy densities of 10⁶ to 10⁸ Joules/m³. Equipment that meet this specification includes but is not limited to ultrasonicators, cavitators, mechanical homogenizers, pressure homogenizers and microfluidizers (Table 2). One such homogenizer is shown in U.S. Pat. No. 756,953, the disclosure of which is incorporated herein by reference. After shear processing, the oxidized carbon nanotubes are discrete and individualized carbon nanotubes. Typically, based on a given starting amount of entangled as-received and as-made carbon nanotubes, a plurality of discrete oxidized carbon nanotubes results from this process, preferably at least about 60%, more preferably at least about 75%, most preferably at least about 95% and as high as 100%, with the minority of the tubes, usually the vast minority of the tubes remaining entangled, or not fully individualized.

Another illustrative process for producing discrete carbon nanotubes follows: A mixture of 0.5% to 5% carbon nanotubes, preferably 3%, by weight is prepared with CNano Flotube 9000 grade carbon nanotubes and an acid mixture that consists of 3 parts by weight of sulfuric acid (97% sulfuric acid and 3% water) and 1 part by weight of nitric acid (65-70 percent nitric acid). The mixture is held at room temperature while stirring for 3-4 hours. The formed oxidized carbon nanotubes are then isolated from the acid mixture. Several methods can be used to isolate the oxidized carbon nanotubes, including but not limited to centrifugation, filtration, mechanical expression, decanting and other solid—liquid separation techniques. The acid is then removed by washing the oxidized carbon nanotubes with an aqueous medium, such as water, preferably deionized water, to a pH of 3 to 4. The oxidized carbon nanotubes are then suspended in water at a concentration of 0.5% to 4%, preferably 1.5% by weight. The solution is subjected to intensely disruptive forces generated by shear (turbulent) and/or cavitation with process equipment capable of producing energy densities of 10⁶ to 10⁸ Joules/m³. Equipment that meet this specification includes but is not limited to ultrasonicators, cavitators mechanical homogenizers, pressure homogenizers and microfluidizers (Table 2). After shear and/or cavitation processing, the oxidized carbon nanotubes become oxidized, discrete carbon nanotubes. Typically, based on a given starting amount of entangled as-received and as-made carbon nanotubes, a plurality of discrete oxidized carbon nanotubes results from this process, preferably at least about 60%, more preferably at least about 75%, most preferably at least about 95% and as high as 100%, with the minority of the tubes, usually the vast minority of the tubes remaining entangled, or not fully individualized.

### EXAMPLE 1

### Entangled Oxidized as MWCNT—3 Hour (oMWCNT-3)

One hundred milliliters of>64% nitric acid is heated to 85 degrees C. To the acid, 3 grams of as-received, multi-walled carbon nanotubes (C9000, CNano Technology) are added. The as-received tubes have the morphology of entangled balls of wool. The mixture of acid and carbon nanotubes are mixed while the solution is kept at 85 degrees C. for 3 hours and is labeled “oMWCNT-3”. At the end of the reaction period, the oMWCNT-3 are filtered to remove the acid and washed with reverse osmosis (RO) water to pH of 3-4. After acid treatment, the carbon nanotubes are still entangled balls. The tubes are dried at 60° C. to constant weight.

### EXAMPLE 2

### Entangled Oxidized as MWCNT—6 Hour (oMWCNT-6)

One hundred milliliters of >64% nitric acid is heated to 85 degrees C. To the acid, 3 grams of as-received, multi-walled carbon nanotubes (C9000, CNano Technology) are added. The as-received tubes have the morphology of entangled balls of wool. The mixture of acid and carbon nanotubes are mixed while the solution is kept at 85 degrees for 6 hours and is labeled “oMWCNT-6”. At the end of the reaction period, the oMWCNT-6 are filtered to remove the acid and washed with reverse osmosis (RO) water to pH of 3-4. After acid treatment, the carbon nanotubes are still entangled balls. The tubes are dried at 60° C. to constant weight.

### EXAMPLE 3

### Discrete Carbon Nanotube—Oxidize Outermost Wall (out-dMWCNT)

In a vessel, 922 kilograms of 64% nitric acid is heated to 83° C. To the acid, 20 kilograms of as received, multi-walled carbon nanotubes (C9000, CNano Technology) is added. The mixture is mixed and kept at 83° C. for 3 hours. After the 3 hours, the acid is removed by filtration and the carbon nanotubes washed with RO water to pH of 3-4. After acid treatment, the carbon nanotubes are still entangled balls with few open ends. While the outside of the tube is oxidized forming a variety of oxidized species, the inside of the nanotubes have little exposure to acid and therefore little oxidization. The oxidized carbon nanotubes are then suspended in RO water at a concentration of 1.5% by weight. The RO water and oxidized tangled nanotubes solution is subjected to intensely disruptive forces generated by shear (turbulent) and/or cavitation with process equipment capable of producing energy densities of 10⁶ to 10⁸ Joules/m³ .The resulting sample is labeled “out-dMWCNT” which represents outer wall oxidized and “d” as discrete. Equipment that meet this shear includes but is not limited to ultrasonicators, cavitators, mechanical homogenizers, pressure homogenizers, and micro fluidizers (Table 2). It is believed that the shear and/or cavitation processing detangles and discretizes the oxidized carbon nanotubes through mechanical means that result in tube breaking and opening of the ends due to breakage particularly at defects in the CNT structure which is normally a 6 member carbon rings. Defects happen at places in the tube which are not 6 member carbon rings. As this is done in water, no oxidation occurs in the interior surface of the discrete carbon nanotubes.

### EXAMPLE 4

### Discrete Carbon Nanotube—Oxidized Outer and Inner WALL (out/in-dMWCNT)

To oxidize the interior of the discrete carbon nanotubes, 3 grams of the out-dMWCNT is added to 64% nitric acid heated to 85° C. The solution is mixed and kept at temperature for 3 hours. During this time, the nitric acid oxidizes the interior surface of the carbon nanotubes. At the end of 3 hours, the tubes are filtered to remove the acid and then washed to pH of 3-4 with RO water. This sample is labeled “out/in-dMWCNT” representing both outer and inner wall oxidation and “d” as discrete.

Oxidation of the samples of carbon nanotubes is determined using a thermogravimetric analysis method. In this example, a TA Instruments Q50 Thermogravimetric Analyzer (TGA) is used. Samples of dried carbon nanotubes are ground using a vibration ball mill. Into a tared platinum pan of the TGA, 7-15 mg of ground carbon nanotubes are added. The measurement protocol is as follows. In a nitrogen environment, the temperature is ramped from room temperature up to 100° C. at a rate of 10° C. per minute and held at this temperature for 45 minutes to allow for the removal of residual water. Next the temperature is increased to 700° C. at a rate of 5° C. per minute. During this process the weight percent change is recorded as a function of temperature and time. All values are normalized for any change associated with residual water removal during the 100° C. isotherm. The percent of oxygen by weight of carbon nanotubes (% Ox) is determined by subtracting the percent weight change at 600° C. from the percent weight change at 200° C.

A comparative table (Table 3 below) shows the levels of oxidation of different batches of carbon nanotubes that have been oxidized either just on the outside (Batch 1, Batch 2, and Batch 3), or on both the outside and inside (Batch 4). Batch 1 (oMWCNT-3 as made in Example 1 above) is a batch of entangled carbon nanotubes that are oxidized on the outside only when the batch is still in an entangled form (Table 3, first column). Batch 2 (oMWCNT -6 as made in Example 2 above) is also a batch of entangled carbon nanotubes that are oxidized on the outside only when the batch is still in an entangled form (Table 3, second column). The average percent oxidation of Batch 1 (2.04% Ox) and Batch 2 (2.06% Ox) are essentially the same. Since the difference between Batch 1 (three hour exposure to acid) and Batch 2 (six hour exposure to acid) is that the carbon nanotubes were exposed to acid for twice as long a time in Batch 2, this indicates that additional exposure to acid does not increase the amount of oxidation on the surface of the carbon nanotubes.

Batch 3 (Out-dMWCNT as made in Example 3 above) is a batch of entangled carbon nanotubes that were oxidized on the outside only when the batch was still in an entangled form (Table 3, third column). Batch 3 was then been made into a discrete batch of carbon nanotubes without any further oxidation. Batch 3 serves as a control sample for the effects on oxidation of rendering entangled carbon nanotubes into discrete nanotubes. Batch 3 shows essentially the same average oxidation level (1.99% Ox) as Batch 1 and Batch 2. Therefore, Batch 3 shows that detangling the carbon nanotubes and making them discrete in water opens the ends of the tubes without oxidizing the interior.

Finally, Batch 4 (Out/In-dMWCNT as made in this Example 4 herein) is a batch of entangled carbon nanotubes that are oxidized on the outside when the batch is still in an entangled form, and then oxidized again after the batch has then been made into a discrete batch of carbon nanotubes (Table 3, fourth column). Because the discrete carbon nanotubes are open ended, in Batch 4 acid enters the interior of the tubes and oxidizes the inner surface. Batch 4 shows a significantly elevated level of average oxidation (2.39% Ox) compared to Batch 1, Batch 2 and Batch 3. The significant elevation in the average oxidation level in Batch 4 represents the additional oxidation of the carbon nanotubes on their inner surface. Thus, the average oxidation level for Batch 4 (2.39% Ox) is about 20% higher than the average oxidation levels of Batch 3 (1.99% Ox). In Table 3 below, the average value of the oxidation is shown in replicate for the four batches of tubes. The percent oxidation is within the standard deviation for Batch 1, Batch 2 and Batch 3.

TABLE 2

| | | Energy |
|---|---|---|
| Homogenizer | | Density |
| Type | Flow Regime | (J-m⁻³) |
| Stirred tanks | turbulent inertial, turbulent viscous, | 10³-10⁶ |
| | laminar viscous | |
| Colloid mil | laminar viscous, turbulent viscous | 10³-10⁸ |
| Toothed - disc | turbulent viscous | 10³-10⁸ |
| disperser | | |
| High pressure | turbulent inertial, turbulent viscous, | 10⁶-10⁸ |
| homogenizer | cavitation inertial, laminar viscous | |
| Ultrasonic probe | cavitation inertial | 10⁶-10⁸ |
| Ultrasonic jet | cavitation inertial | 10⁶-10⁸ |
| Microfluidization | turbulent inertial, turbulent viscous | 10⁶-10⁸ |
| Membrane and | Injection spontaneous | Low 10³ |
| mircochannel | transformation based | |

Excerpted from Engineering Aspects of Food Emulsification and Homogenization, ed. M. Rayner and P. Dejmek, CRC Press, New York 2015.

TABLE 3
Percent oxidation by weight of carbon nanotubes.

| | | | Batch 3: | Batch 4: | Difference | *% |
|---|---|---|---|---|---|---|
| | Batch 1: | Batch 2: | Out- | Out/In- | in % Ox | difference in |
| | oMWCNT-3 | oMWCNT-6 | dMWCNT | dMWCNT | (Batch 4 − | % Ox (Batch |
| | % Ox | % Ox | % Ox | % Ox | Batch 3) | 4 v Batch 3) |
| | 1.92 | 1.94 | 2.067 | 2.42 | 0.353 | 17% |
| | 2.01 | 2.18 | 1.897 | 2.40 | 0.503 | 26.5% |
| | 2.18 | NM | 2.12 | 2.36 | 0.24 | 11% |
| | 2.05 | NM | 1.85 | NM | n/a | n/a |
| Average | 2.04 | 2.06 | 1.99 | 2.39 | 0.4 | 20% |
| St. Dev. | 0.108 | 0.169 | 0.130 | 0.030 | n/a | n/a |

NM = Not Measured
*% difference between interior and exterior oxidation surfaces (Batch 4 v Batch 3) = (((outside % oxidation) − (inside % oxidation)) ÷ (outside % oxidation)) × 100

Disclosed embodiments may also relate to a composition useful for targeted delivery of drugs, chemicals, compounds, and/or small molecules. Embodiments may also relate to directing the controlled release or adjusting the breakdown or clearance of drugs, chemicals, compounds, and/or small molecules.

Embodiments may also relate to treating and/or remediating contaminated soil, groundwater and/or wastewater by treating, removing, modifying, sequestering, targeting labeling, and/or breaking down at least a portion of any dry cleaning compounds and related compounds such as perchloroethene (PCE), trichloroethene (TCE), 1,2-dichloroethene (DCE), vinyl chloride, and/or ethane. Embodiments may also relate to compounds useful for treating, removing, modifying, sequestering, targeting labeling, and/or breaking down at least a portion of any oils, hazardous or undesirable chemicals, and other contaminants. Disclosed embodiments may comprise a plurality of discrete carbon nanotubes, wherein the discrete carbon nanotubes comprise an interior and exterior surface. Each surface may comprise an interior surface oxidized species content and/or an exterior surface oxidized species content. Embodiments may also comprise at least one biologically or chemically active molecule that is attached on either the interior or the exterior surface of the plurality of discrete carbon nanotubes. Such embodiments may be used in order to deliver known biologically and/or chemically active molecules to a desired location within the body and/or to maintain such biologically and/or chemically active molecules at a desired location once delivered.

Rubber Experiment

A composition of individual carbon nanotubes in an elastomer matrix can be achieved by using the following method:

Raw CNTs purchased on the market, such as C9000 from CNano, are mixed into de-ionized water until a suspension of the CNT bundles are formed. A typically-used process oil for rubber formulations, such as naphthenic oil HyPrene L150 from Ergon International, is then added to the suspension. The CNTs then transfer from the aqueous phase into the oil phase thru a high shear mixing process, such as an overhead stirrer using a Cowles style blade. Depending on the concentration of CNT to oil, a powder, cake, or liquid can be formed. The CNT/Oil mixture is then dried of all remaining water through typical means, such as a convection oven, resulting in a pure CNT/Oil mixture. This mixture, along with other rubber compounding ingredients, such as typically used carbon blacks in rubber formulations, are added into a typical rubber processing shear device, such as a tangential mixer, inter-meshing blade mixer, 2 roll mill, calendar, or extruder, disperses in a usual mix cycle, typically ˜4 minutes of total mixing. These CNTs are individualized in the rubber mixing process, resulting in a reinforced matrix that could have improved tear resistance properties, electrical and thermal conductivities, modulus values, and a higher viscosity.

Addition of Payload Molecules

Aqueous solubility of drug substances is an important parameter in pre-formulation studies of a drug product. Several drugs are sparingly water-soluble and pose challenges for formulation and dose administration. Organic solvents or oils and additional surfactants to create dispersions can be used. If the payload molecule is easily dissolved or dispersed in an aqueous media, the filter cake need not be dried. If the payload molecule is not easily dissolved or dispersed in aqueous media, the filter cake is first dried at 80° C. in vacuo to constant weight. The payload molecule in the liquid media at the desired concentration is added to the discrete carbon nanotubes and allowed several hours to equilibrate within the tube cavity. The mixture is then filtered to form a cake, less than about 1 mm thickness, then the bulk of the payload solution not residing within the tubes are removed by high flow rate filtration. The rate of filtration is selected so that little time is allowed for the payload molecules to diffuse from the tube cavity. The filter cake plus payload drug is then subjected to an additional treatment if desired to attach a large molecule such an aqueous solution of a biopolymer, an amino acid, protein or peptide.

### EXAMPLE 5

A calibration curve for the UV absorption of niacin as a function of the concentration of niacin in water was determined. A solution was prepared by mixing 0.0578 grams of discrete functionalized carbon nanotubes of this invention with 0.0134 grams of niacin in 25 ml of water [0.231 grams niacin/gram of carbon nanotube]. The tubes were allowed to settle and an aliquot of the fluid above the tubes removed hourly. The UV-vis absorption of this aliquot was measured and the resulting amount of niacin in the solution recorded. The amount of niacin in solution stabilized after 6 hours. A final sample was taken 20 hours after mixing. The difference between the amounts of niacin remaining in the solution and the original amount was determined to be the amount of niacin associated with the discrete functionalized carbon nanotubes. It was found that 0.0746 grams of niacin associated with each gram of carbon nanotubes. The total amount of niacin absorbed by the carbon nanotubes was 0.0043 grams. Assuming an average carbon nanotube length of 1,000 nm, external diameter of 12 nm and internal diameter of 5 nm, the available volume within the tube is 0.093 cm³ per gram of carbon nanotubes. Since the density of niacin is 1.473 g/cm³, then the maximum amount of niacin that can fit in the tubes is 0.137 grams. Therefore, the measured absorption of 0.0746 g niacin/g CNT amount could be confined to the interior of the tube.

### EXAMPLE 6

A poly (vinyl alcohol), PVOH, is sufficiently large (30 kDa-70 kDa) that it cannot be absorbed internally in a carbon nanotube. PVOH is used as a surfactant for carbon nanotubes because it associates and wraps the exterior of the carbon nanotube. In this experiment, PVOH was added to a mixture of 0.0535 g of carbon nanotubes and 0.0139 g niacin (0.26 grams niacin to 1 gram carbon nanotubes) in 25 ml water. This was allowed to rest overnight. Using the UV-vis technique of Example 5, the amount of niacin associated with the carbon nanotubes was determined to be 0.0561 grams niacin per gram of carbon nanotubes, less than the 0.0746 grams in Example 5. The total amount of niacin absorbed was 0.003 grams.

Calculations were made assuming carbon nanotube length of 1,000 nm, external diameter of 12 nm and internal diameter of 5 nm. Given the density of PVOH is 1.1 g/cm³ and the ratio of PVOH to carbon nanotubes was 0.23 to 1, the average layer thickness of PVOH on the carbon nanotube is 0.6 nm. Therefore there is sufficient PVOH to encapsulate the carbon nanotube and displace any niacin on the surface of the tube and the measured amount of 0.0561 grams of niacin per gram of carbon nanotubes is in the interior of the carbon nanotube.

Modulating BioDistribution and Controlled Release of Drug Loaded MR

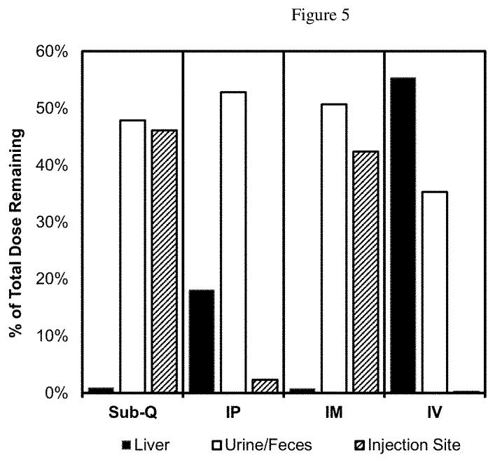

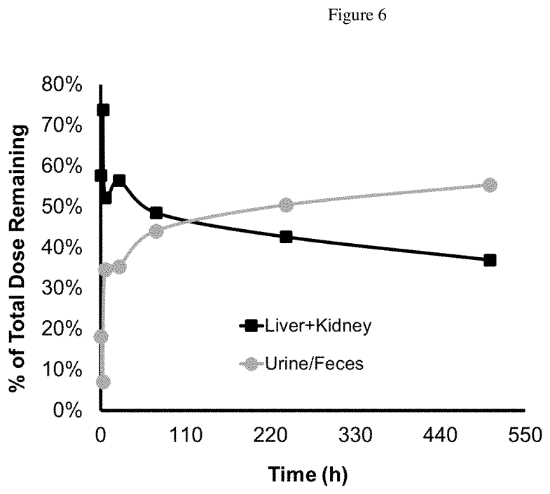

The route of administration may have an impact on the biodistribution of the disclosed compositions. FIG. 5 shows the percent of total dose remaining 24 hours after administration for various administration methods. FIG. 6 shows the clearance of the disclosed nanotubes through multiple pathways over time.

In some embodiments, the nanotubes described herein have altered surface chemistry which allows for control of the biodistribution of nanotubes. This is particularly useful for controlling the biodistribution of loaded nanotubes, preferably drug-loaded nanotubes. Altering the biodistribution of drug-loaded nanotubes allows for improved dose response by controlling the amount of drug accumulation in targeted tissues. In preferred embodiments, the target tissue is liver, bone, and/or blood.

Nanotube surface chemistry may be altered by functionalizing the surface and/or coating the nanotubes. In some embodiments, PEG is used to functionalize the nanotube surface. The density and type of functionalization used, including the type of terminal group and/or terminal charge of any species attached to a nanotube influence the biodistribution of nanotubes.

In certain embodiments, PEG is covalently linked to the surface of the nanotubes. In particular embodiments, PEG is covalently attached to the surface of nanotubes using a thionyl chloride addition between the hydroxyl group of the PEG polymer and the carboxylic acid groups of an oxidized nanotube. In some embodiments, controlling the degree and location of nanotube oxidation allows for controlling of the degree and location of surface coating. In other embodiments, altering the surface chemistry of the nanotubes may be accomplished using a bio-compatible polymer or functionalizing agent other than PEG.

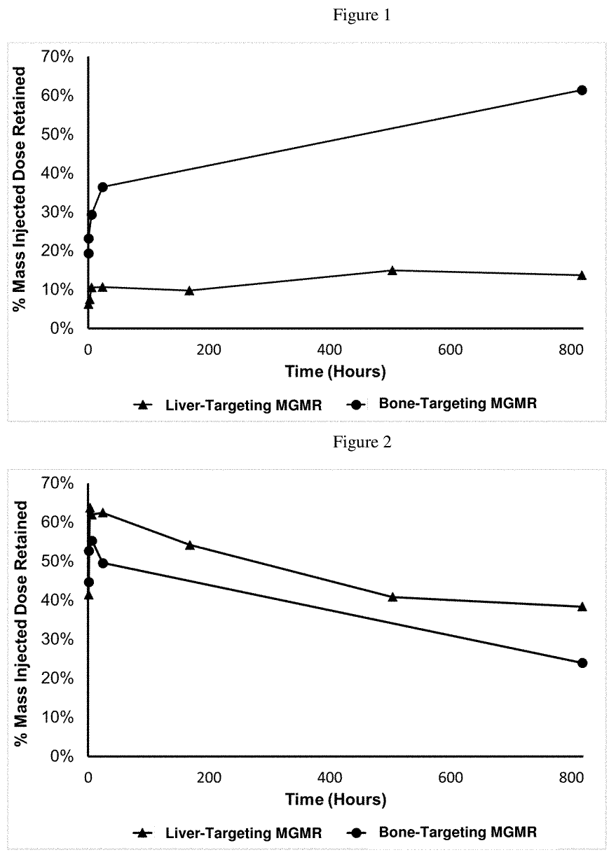

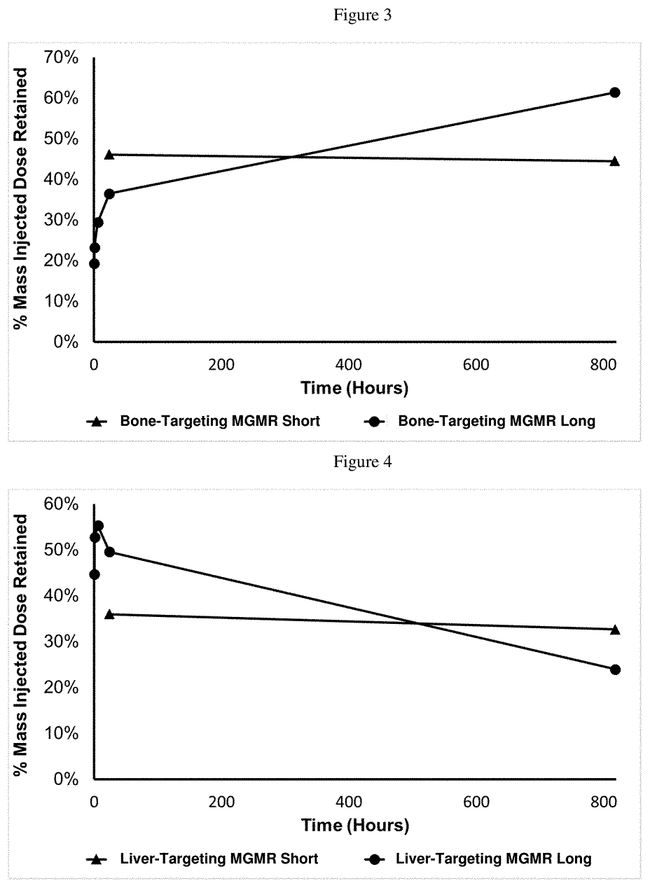

In other embodiments, amphiphilic Poly(ethylene glycol) surfactant (DSPE-PEG) may, additionally or alternatively, be noncovalently attached to the nanotube surface through hydrophobic interactions between a phospholipid chain and hydrophobic pockets found on the surface of the nanotubes which are void of oxidation. Changing the surfactant ratio or terminal functional group of the PEG has been shown to cause changes in the biodistribution of the PEG-MR complex. Merely as one example, changing from a methyl terminated PEG to a primary amine terminated PEG has been shown to cause a large change in biodistribution. As can be seen in FIGS. 1 and 2, when this change was tested, liver retention decreased and bone accumulation increased nearly 4 times. In FIGS. 1 and 2, the change from 2 to 3+ involved the addition of a primary amine, instead of a methyl group, to the DSPE-PEG surfactant which was used to disperse and functionalize the MR.

Specific ranges of PEG density on the nanotube surface are required for biocompatibilization and may assist in maintaining nanotubes in a discrete form in the blood and tissues. In some embodiments, the w/w range of PEG:MR of covalently linked PEG may be as low as about 1%, about 3%, about 5%, about 7%, about 8%, about 8.5%, about 9%, about 9.5%, or about 10%. The w/w range of PEG:MR of covalently linked PEG may be as high as about 10%, about 10.5%, about 11%, about 11.5%, about 12%, about 13%, about 15%, about 17%, or about 19%. In some embodiments, the weight ratios of PEG surfactant to MR of non-covalently attached PEG surfactant may be as low as about 0.01:1, about 0.05:1, about 0.1:1, about 0.15:1, about 0.2:1, about 0.25:1, about 0.3:1 or about 0.4:1. In some embodiments, the weight ratios of PEG surfactant to MR of non-covalently attached PEG surfactant may be as high as about 0.5:1, about 0.6:1, about 0.7:1, about 0.75:1, about 0.8:1, about 0.85:1, about 0.9:1, or about 1:1.

The process of forming MR/PEG dispersions may also be modified to control the type of drug-loading. Adding a desired drug and PEG to an MR sample in a single step or forming drug-encapsulating micelles prior to loading allows for the formation of drug-loaded micelles which may associate with the MR surface in some embodiments. This type of drug-loading may produce different off-loading characteristics and release kinetics relative to drug loaded on the MR following treatment with PEG.

Molecules which undergo chemical or physical changes in response to changes in physiological conditions (temperature, ionic concentration, pH) may be incorporated on MR surface in order to further regulate delivery. For example, a pH-sensitive polymer which decomposes at acidic pH (below 7.4) may allow for selective delivery of drugs to acidic or tumor like environments.

In some embodiments, zwitterionic molecules, including but not limted to carboxybetaine phosphoryl choline, and/or polymers therefrom, may be employed to regulate drug delivery: drug-loading, biodistribution, tissue/organ targeting, drug off-loading and/or clearance.

In some embodiments, chemicals, compounds, and/or small molecules, useful for scanning, imaging and/or diagnostics may be loaded onto MR alternatively or in addition to drugs, chemicals, compounds, and/or small molecules. These embodiments may be useful for monitoring, confirming, and/or quantizing the delivery of drug to a particular target.

Controlling Biodistribution by Utilization of Nanotubes of Preferred Length

In some embodiments, the collections of nanotubes described herein comprise approximately normal distributions of length-limited nanotubes, which allows for control of the bioaccumulation and/or clearance of nanotubes from specific tissues, organs and/or cells residing therein. This is particularly useful for controlling the biodistribution of loaded nanotubes, preferably drug-loaded nanotubes in order to increase or decrease the total concentration of nanotubes in a given tissue, organ or collection of cells, or the rate by which the nanotubes accumulate or are cleared therefrom.

Regular Tubes: Prepared by oxidation in acid. The resulting nanotubes were characterized by an approximately normal distribution of 850 nm average length nanotubes.

Short Tubes: Short tubes were prepared by an extended oxidation in acid. Material was washed extensively with DI water to pH 4. The resulting nanotubes were characterized by an approximately normal distribution of 450 nm average length nanotubes.

Short and Regular PEGylated tubes: Tubes were functionalized via nucleophilic acyl addition/elimination. Briefly, tubes were mixed in anhydrous DMF with thionyl chloride for one hour to convert carboxylic acids to an acyl chloride. Following this conversion, tubes were mixed overnight with mono-functional hydroxyl polyethylene glycol (5k mW). These tubes were then dispersed using DSPE-PEG at a weight ratio of 0.7:1 surfactant to tubes using a water bath sonicator for one hour.

All tubes: Tubes underwent Williamson ether reaction utilizing 3-chloropropylamine (CPA). Briefly, a 10× molar excess of CPA was mixed into DI water and heated to 55C. CNT was added to the mixing solution to end up with a 1% w/v solution of CNT. Solution was titrated to pH 11 with NaOH and repeatedly titrated to maintain pH above 11. Reaction was stopped once pH remained at steady level for 30 min. Solution was washed extensively with DI water to remove any unreacted material.

Utilizing CNT-NH2, p-SCN-Bn-DOTA (DOTA isothiocyanate) was attached by reacting of the isothiocyante group to the amine of the tubes to form a thiourea bond by mixing the two at pH 9 for one hour. Unreacted material was removed via extensive washing using centrifugal tubes.

Lu177 radioisotope was chelated onto the tubes via DOTA functional groups. This was accomplished by mixing solution of Lu177 with CNT-DOTA for a 4-hour incubation. Excess radiolabel was removed by extensive washing using a 10mM solution of EDTA.

Dispersions Short and Regular CNTs (DSPE-PEG, short with DSPE-PEG) were injected into mice via tail vein. At designated times points, 3 mice were sacrificed and internal organs were removed and weighed. Organs, carcass and feces/urine were analyzed via scintillation counting to determine distribution of isotope (%ID, %ID/g) throughout the mice.

### Embodiments

Embodiments disclosed in this application include at least:
1. A composition comprising a plurality of discrete carbon nanotubes, wherein the discrete carbon nanotubes comprise an interior and exterior surface, the interior surface comprising an interior surface oxidized species content and the exterior surface comprising an exterior surface oxidized species content, wherein the interior surface oxidized species content comprises from about 0.01 to less than about 4 percent relative to carbon nanotube weight and the exterior surface oxidized species content comprises more than about 1 to about 10 percent relative to carbon nanotube weight, wherein a biocompatible surface coating is attached to at least a portion of the exterior surface of the discrete carbon nanotubes or of the interior of the discrete carbon nanotube.
2. The composition of embodiment 1, wherein the biocompatible surface coating is selected from the group consisting of PEG (polyethylene glycol), PLA (polylactic acid), PVOH (polyvinyl alcohol), PEO (polyethylene oxide), PGLA (polyglycolic acid), CMC (carboxymethyl cellulose), PVP (polyvinylpyrrolidone), PAA (polyacrylic acid), aminoacids, peptides, polysaccharides and proteins.
3. The composition of embodiment 1, wherein the biocompatible surface coating is a poly ethylene glycol.
4. The composition of embodiment 3, wherein the terminal group of the poly ethylene glycol is a methyl group.
5. The composition of embodiment 3, wherein the terminal group of the poly ethylene glycol is a primary amine.
6. The composition of embodiment 3, wherein the poly ethylene glycol is covalently linked to the exterior surface.
7. The composition of embodiment 3, wherein the poly ethylene glycol is a surfactant and is non-covalently linked to the exterior surface.
8. The composition of embodiment 6, wherein the w/w range of poly ethylene glycol to discrete carbon nanotubes is between about 7% to about 13%, preferably between about 9% to about 11%.
9. The composition of embodiment 7, wherein the weight ratio of poly ethylene glycol surfactant to discrete carbon nanotubes is between about 0.05:1 to about 1:1, preferably between about 0.2:1 to about 0.8:1.
10. The composition of embodiment 1, further comprising at least one type of payload molecule.
11. The composition of embodiment 10, wherein the payload molecule is attached to the exterior surface of the discrete carbon nanotubes.
12. The composition of embodiment 10, wherein the payload molecule is a drug-encapsulating micelle
13. The composition of embodiment 10, wherein the payload molecule has a molecular weight of less than about 10,000 Daltons.
14. The composition of embodiment 1, further comprising at least one imaging molecule for determining the location of the discrete carbon nanotubes.
15. The composition of embodiment 1, further comprising a pH sensitive polymer attached to the exterior surface of the discrete carbon nanotubes.
16. The composition of embodiment 1, further comprising electromagnetic species such as iron or its oxides attached to the exterior surface of the discrete carbon nanotubes.
17. The composition of embodiment 1, wherein the biocompatible surface coating has a molecular weight greater than about 30,000 Daltons.
18. A payload molecule delivery system composition comprising discrete oxidized carbon nanotubes, at least one payload molecule, and at least one type of biocompatible surface coating, wherein the distribution of aspect ratios of the discrete carbon nanotubes is bimodal.
19. A process useful for dispersing carbon nanotubes into a polymer, comprising of (a) soaking and agitating entangled carbon nanotubes in an aqueous solution at a temperature above 25° C., (b) phase transferring the carbon nanotubes to a new medium by mixing at high shear and elevated temperature, (c) removing excess water, and (d) mixing into a final polymer formulation using high shear compounding equipment.
20. The process in embodiment 19, wherein the steps are sequential.
21. The process in embodiment 19, wherein the entangled carbon nanotubes in step (a) are commercially available and have not been physically or chemically altered in any way prior to the described process.
22. The process in embodiment 19, wherein the elevated temperature in step (a) is preferentially between 35-100° C., and especially between 55-75° C.
23. The process in embodiment 19, wherein the agitating in step (a) is performed using a high shear mixer.
24. The process in embodiment 19, wherein the phase transfer from aqueous medium to new medium in step (b) takes place on a shear-dependent time scale, such that higher shear corresponds to shorter process time.
25. The process in embodiment 19, wherein the phase transfer from aqueous medium to new medium at elevated temperature in step (b) takes place in a preferential temperature range of 35-100° C., with the most preferential range between 55-75° C.
26. The process in embodiment 19, wherein the new medium in step (b) is a commonly used processing aid or ingredient in the rubber compounding industry, such as but not limited to: Trioctyl Trimellitate (TOTM), Dioctyl Adipate (DOA), Dibutoxyethoxyethyl adipate, castor oil, naphthenic oil, residual aromatic extract oil (RAE), treated distillate aromatic extracted oil (TDAE), aromatic oils, paraffinic oils, carnauba wax, curing co-agents, natural waxes, synthetic waxes, and peroxide curatives.
27. The process in embodiment 19, wherein the polymer in step (d) is selected from the following group: plastics, elastomeric polymers, synthetic rubbers, natural rubbers, hydrocarbon-based polymers, and blends of afore mentioned polymers.
28. The process in embodiment 19, wherein the final polymer formulation in step (d) has a filler content higher than 15 parts per hundred rubber, with a preferential range between 20 and 90 parts per hundred rubber.
29. The process in embodiment 19, wherein the high shear compounding equipment in step (d) exerts a shear force on the compound
30. The process in embodiment 19, wherein the high shear compounding equipment is selected from the following list: tangential type mixer, intermeshing type mixer, 2 roll mill, calendaring mill, screw type extruder, or some processing combination utilizing one or more of these compounding equipment.

What is claimed is:

1. A composition comprising a plurality of discrete carbon nanotubes, wherein the discrete carbon nanotubes comprise an interior and exterior surface, the interior surface comprising an interior surface oxidized species content and the exterior surface comprising an exterior surface oxidized species content, wherein the interior surface oxidized species content comprises from about 0.01 to less than about 1 percent relative to carbon nanotube weight and the exterior surface oxidized species content comprises more than about 1 to about 10 percent relative to carbon nanotube weight, wherein a biocompatible surface coating is attached to at least a portion of the exterior surface of the discrete carbon nanotubes.

2. The composition of claim 1, wherein the biocompatible surface coating is derived from a precursor selected from PEG (polyethylene glycol), PLA (polylactic acid), PVOH (polyvinyl alcohol), PEO (polyethylene oxide), PGLA (polyglycolic acid), CMC (carboxymethyl cellulose), PVP (polyvinylpyrrolidone), PAA (polyacrylic acid), aminoacids, peptides, polysaccharides, nucleic acids and proteins.

3. The composition of claim 1, wherein the biocompatible surface coating is derived from a polyethylene glycol precursor.

4. The composition of claim 1, wherein the biocompatible surface coating is derived from CRISPR/Cas9-based gene editing technology.

5. The composition of claim 1, wherein the biocompatible surface coating is derived from biomolecular components of zinc finger nuclease- or transcription activator-like effector nuclease-based gene editing technology.

6. The composition of claim 1, wherein the biocompatible surface coating is derived from a carboxy betaine precursor.

7. The composition of claim 1, wherein the biocompatible surface coating is derived from a phosphoryl choline precursor.

8. The composition of claim 1, wherein the biocompatible surface coating is derived from a zwitterionic moiety.

9. The composition of claim 3, wherein the polyethylene glycol precursor comprises a methyl terminal group.

10. The composition of claim 3, wherein the polyethylene glycol precursor comprises a primary amine terminal group.

11. The composition of claim 3, wherein the polyethylene glycol precursor is covalently attached to the exterior surface.

12. The composition of claim 3, wherein the polyethylene glycol precursor is a surfactant and is non-covalently attached to the exterior surface.

13. The composition of claim 12, wherein a weight ratio of polyethylene glycol to discrete carbon nanotubes is between about 7% to about 13%.

14. The composition of claim 12, wherein a weight ratio of polyethylene glycol surfactant to discrete carbon nanotubes is between about 0.05:1 to about 1:1.

15. The composition of claim 1, further comprising at least one type of payload molecule.

16. The composition of claim 15, wherein the payload molecule is attached to the exterior surface or interior or both of the discrete carbon nanotubes.

17. The composition of claim 15, wherein the payload molecule comprises an organic or inorganic nanoparticle.

18. The composition of claim 15, wherein the payload molecule comprises a drug-encapsulating micelle.

19. The composition of claim 15, wherein the payload molecule has a molecular weight of less than about 10,000 Daltons.

20. The composition of claim 1, further comprising at least one imaging molecule for determining the location of the discrete carbon nanotubes.

21. The composition of claim 1, further comprising a pH sensitive polymer attached to the exterior surface of the discrete carbon nanotubes or to the biocompatible surface coating.

22. The composition of claim 1, wherein the biocompatible surface coating has a molecular weight greater than about 30,000 Daltons.

23. The composition of embodiment 1, further comprising electromagnetic species such as iron or its oxides attached to the exterior surface of the discrete carbon nanotubes.

24. A payload molecule delivery system composition comprising discrete oxidized carbon nanotubes, at least one type of payload molecule, and at least one type of biocompatible surface coating, wherein the at least one type of biocompatible surface coating is covalently or non-covalently attached to at least a portion of the exterior surface of the discrete carbon nanotubes and wherein the at least one type of payload molecule is covalently or non-covalently attached to the biocompatible surface coating.

25. The payload molecule delivery system of claim 24 wherein the molecular surface coating or payload comprises the group consisting of: small molecules, surfactants, polymers, composites, organic and inorganic nanoparticles, peptides, proteins, enzymes, nucleic acids, oligonucleotides, carbohydrates, lipids, glycosaminoglycans, proteoglycans, glycoproteins, steroids, antibodies, growth factors, viral components, viral vectors, genetic materials, cell-derived components and macromolecular gene-editing assemblies, other biologics and complexes thereof.

26. The payload molecule delivery system of claim 24 wherein a distribution of aspect ratios of the discrete carbon nanotubes is bimodal.

27. The payload molecule delivery system of claim 24 wherein at least one type of the biocompatible surface coating directs the biological distribution nanotubes to organs, tissues and/or cells residing therein, within an organism.

28. The payload molecule delivery system of claim 24 with a preferred distribution of average lengths of the discrete nanotubes is from about 800 nm to about 10 nm.

29. A payload molecule delivery system composition comprising discrete, oxidized carbon nanotubes, where one or more payload molecules is attached or adsorbed to at least a portion of the exterior surface of the discrete carbon nanotubes.

30. The payload molecule delivery system of claim 29 wherein the payload is selected from the group consisting of small molecules, surfactants, polymers, composites, organic and inorganic nanoparticles, peptides, proteins, enzymes, nucleic acids, oligonucleotides, carbohydrates, lipids, glycosaminoglycans, proteoglycans, glycoproteins, steroids, antibodies, growth factors, viral components, viral vectors, genetic materials, cell-derived components and macromolecular gene-editing assemblies, other biologics and complexes thereof.

31. The payload molecule delivery system of claim 29 comprising a distribution of average lengths of the discrete nanotubes ranges between 800 nm and 10 nm.

32. A composition comprising a plurality of discrete carbon nanotubes, wherein the discrete carbon nanotubes comprise an interior and exterior surface, the interior surface comprising an interior surface oxidized species content and the exterior surface comprising an exterior surface oxidized species content, wherein the interior surface oxidized species content comprises from about 0.01 to less than about 1 percent relative to carbon nanotube weight and the exterior surface oxidized species content comprises more than about 1 to about 10 percent relative to carbon nanotube weight, wherein a biocompatible surface coating is attached to at least a portion of the interior surface of the discrete carbon nanotubes.
